(19) United States
(12) Patent Application Publication
(10) Pub. No.: US 2024/0423870 A1
(43) Pub. Date: Dec. 26, 2024

(54) IMPROVED AUTOMATED MECHANICAL VENTILATORS AND NEW METHODS OF USING SAME

(71) Applicant: Spiro Health LLC, Brooklyn, NY (US)

(72) Inventors: Marcel Botha, Brooklyn, NY (US); Nevan Clancy Hanumara, Kingston, RI (US); Michael DeTienne, Cambridge, MA (US); Albert Kwon, San Jose, CA (US); Alexander H. Slocum, JR., Exeter, NH (US); Jan Du Preez, Johannesburg (ZA)

(73) Assignee: Spiro Health LLC, Brooklyn, NY (US)

(21) Appl. No.: 18/756,458

(22) Filed: Jun. 27, 2024

Related U.S. Application Data
(63) Continuation of application No. PCT/US24/34694, filed on Jun. 20, 2024.
(60) Provisional application No. 63/509,199, filed on Jun. 20, 2023.

(51) Int. Cl. A61H 31/00; A61M 16/00; A61M 16/10

(52) U.S. Cl. CPC A61H 31/006; A61M 16/0063; A61M 16/1005; A61H 2201/107; A61H 2201/1215; A61M 2016/0027; A61M 2205/103; A61M 2205/3379; A61M 2230/43

(57) ABSTRACT

Portable automated mechanical ventilators and new methods of using same are disclosed. A resuscitator bag is suspended horizontally via bag mounts and compressed and decompressed by paddles, which may be foldable to increase portability, and the resuscitator bag may be releasable for manual operation. The portable mechanical ventilator may automatically adjust ventilation based on measured parameters to improve safety and be integrated into a CPR workflow.

### I. PRIORITY CLAIM

The present invention claims priority from U.S. Provisional Application No. 63/509,199, filed Jun. 20, 2023, the disclosure of which is hereby incorporated by this reference in its entirety.

### II. FIELD OF INVENTION

The invention disclosed herein relates to improved automated mechanical ventilators and new methods of utilizing such ventilators including integrating the ventilator into a conventional CPR workflow and implementing safe envelopes for operation of a ventilator.

### III. BACKGROUND OF THE INVENTION

When first responders in the field encounter a patient who cannot breathe on their own, hospital-grade mechanized ventilators are neither a practical nor available solution, in part as a result of their size and infrastructure requirements. To ventilate patients in “pre-hospital” settings (such as the scene of an accident, a field or combat hospital, or in transit to a hospital), health professionals will use a “bag valve mask” that requires manual compression to deliver oxygen into the patient's lungs, where each squeeze of the bag acts as a substitute for a single breath. The need for pre-hospital ventilation is particularly acute in developing countries, where there are anecdotal accounts of extended bagging while in transit from remote locations.

Manual bagging saves lives, but it is not a substitute for a hospital grade ventilator. Too forceful a manual compression can overinflate a patient's lungs and damage the alveoli that absorb and transmit oxygen into the bloodstream. Insufficient compression, or compression at a frequency that is too slow, risks providing too little oxygen to a patient, which can result in brain damage or death. Hospital ventilators offer superior consistency and safety with respect to the rate of airflow of each breath (called the “breath profile”) and the frequency of breaths, but are significantly bulkier and more expensive than bag valve mask, precluding their use outside of hospitals in developed countries. Portable mechanical ventilators combine the portability and low cost of manual resuscitators with the safety and other features of hospital ventilators.

Currently, however, portable mechanical ventilators suffer from their own shortcomings. Users selecting portable mechanical ventilator must make tradeoffs with respect to cost, weight/portability, safety features, device precision, component reusability, repairability, and the user experience (including operation by non-medical professionals). Full-featured electric portable ventilators are bulky, expensive, mechanically complex, and have limited battery life. Smaller electric portable ventilators and pneumatic ventilators offer a one-size-fits-all breath profile and have limited features.

An inexpensive, portable resuscitator with automated and user-adjustable settings can address the shortcomings of emergency resuscitators, hospital ventilators, and current portable mechanical ventilators.

The foregoing examples of the related art and limitations thereof are intended to be illustrative and not exclusive. Other limitations of the related art will become apparent to those of skill in the art upon a reading of the specification.

### IV. BRIEF SUMMARY OF THE INVENTION

The present disclosure provides automated mechanical ventilators and new methods of using automated mechanical ventilators. A resuscitator bag is suspended horizontally via bag mounts and compressed and decompressed by paddles.

In accordance with one aspect of the present invention, the arm and paddle assembly are foldable. In another aspect the bag mounts are adjustable to accommodate different bags and changes in the dimensions of the resuscitator bag while the device is in operation. In another aspect, the shape of the paddles is optimized to minimize or eliminate mechanical stresses on the resuscitator bag. In another aspect, the bag restraints are releasable under forces that exceed normal operating forces. In some embodiments, the ventilator may shape an artificial breath profile by modulating the rate of flow through the gas output port into the patient's lungs. New actuation mechanisms for the paddle and arm assembly are also described. In some embodiments, the ventilator may determine the force exerted by the paddles on the bag or may determine mechanical fault conditions by measuring the current used by the motor actuating the paddle motion.

The present disclosure also describes methods of integrating an automated ventilator into a CPR workflow and additional safety features.

### V. BRIEF DESCRIPTION OF THE DRAWINGS

FIG. 1 shows an overview of one embodiment of the inventive automated mechanical ventilator.

FIG. 2 shows an alternative embodiment of the inventive automated mechanical ventilator of the instant disclosure.

FIG. 3 shows additional details of the embodiment of the instant disclosure.

FIG. 4a depicts an embodiment in which the paddle may be folded alongside the enclosure.

FIG. 4b depicts one embodiment of folding joint in which a spring-loaded pull knob may be used to release the arm.

FIG. 5a depicts an exemplary embodiment in which the paddles may serve as carrying handles for the device by means of a protrusion on the side of the paddle not facing the bag.

FIG. 5b depicts exemplary embodiments in which the paddles may serve as carrying handles for the device by means of cutouts through the paddles.

FIG. 6a depicts an exemplary embodiment in which the bag mounts may be adjusted to accommodate bags of different dimensions and shows a first bag with a first geometry.

FIG. 6b depicts an exemplary embodiment in which the bag mounts may be adjusted to accommodate bags of different dimensions and shows a second bag with a second geometry.

FIG. 7 depicts a paddle design that reduces wear on the bag.

FIG. 8a depicts an embodiment of the device in which the bag restraints release under excess force where the bag is secured by bag restraints as if under normal operation.

FIG. 8b depicts an embodiment of the device in which the bag restraints release under excess force where an excess force has been applied to the bag and the bag restraint has shifted or translated to allow one end of the bag to be removed from the device.

FIG. 8c depicts an embodiment of the device in which the bag restraints release under excess force and shows a closer depiction of the shift or translation of the bag restraint under excess force.

FIG. 9a shows an exemplary actuation mechanism of the device.

FIG. 9b shows the exemplary actuation mechanism of the preceding figure from above.

FIG. 10 illustrates exemplary embodiments in which the ventilator is in communication with one or more external devices.

FIG. 11 illustrates an exemplary embodiment in which the ventilator may be connected to a patient transport device.

FIG. 12 illustrates exemplary embodiments in which the ventilator is configured with mechanical attachments to various devices.

### VI. DETAILED DESCRIPTION

### A. Description of the Device

### 1. Overview of Mechanical Design

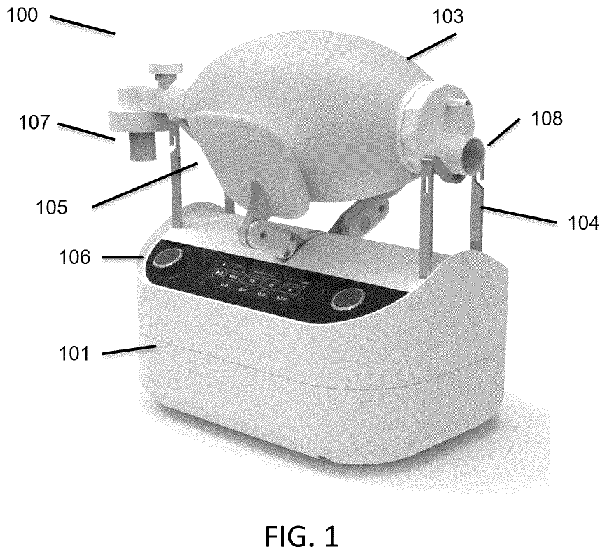

As illustrated in FIG. 1, various embodiments of the present disclosure comprise a portable, electric, automated mechanical ventilator with user-configurable behavior, allowing emergency respiratory resuscitation and respiratory ventilation in a much smaller form factor than traditional hospital ventilators, with features unavailable in current portable resuscitators and ventilators.

In one embodiment, a portable ventilator apparatus and system 100 of the present disclosure includes an electronic and mechanical systems enclosure 101 over which a resuscitator bag 103 is suspended horizontally via bag mounts 104. Electrical and mechanical assemblies inside the system's enclosure actuate paddles 105 that compress and decompress the resuscitator bag. The control panel 106 may allow a user to input relevant settings and, in some embodiments, set rates and frequencies of compressions. Particular operational settings may also be determined algorithmically in whole or part. When compressed, the resuscitator bag delivers oxygen to a patient via the gas output port 107, and when decompressed, the resuscitator bag draws in oxygen from a separate oxygen gas supply via the gas input port 108.

In some embodiments, the surfaces of the portable ventilator apparatus and system of the present disclosure are smooth and the edges are rounded so that the apparatus and system can be easily wiped clean and sanitized if exposed to dirt, debris or bodily fluids. The portable ventilator apparatus and system of the present disclosure may operate with a range of power sources, such as 110/240V AC or 12V DC, and may also include a swappable battery that can be charged onboard or externally.

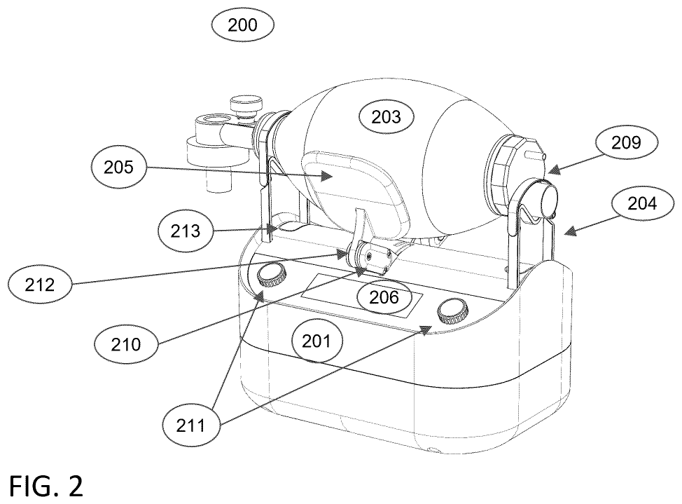

FIG. 2 shows other aspects of an embodiment of the present disclosure. In FIG. 2, the bag 203 is positioned above the device enclosure 201 body between a pair of articulating paddles 205. Paddle arms 210 and adjustable height bag holders 204 support the bag, which is held in place on the bag holders by bag securements 209. The bag holders extension and retraction is governed by a release button 213. In one embodiment, the user interface consists of a combination of a touch display and a manual combined rotary pushbutton to allow intuitive selection (via the touchscreen), adjustment of values (via the dial) and confirmation of selection (via a button). Additionally, the screen may provide system status information.

### 2. Folding Paddles and Arms

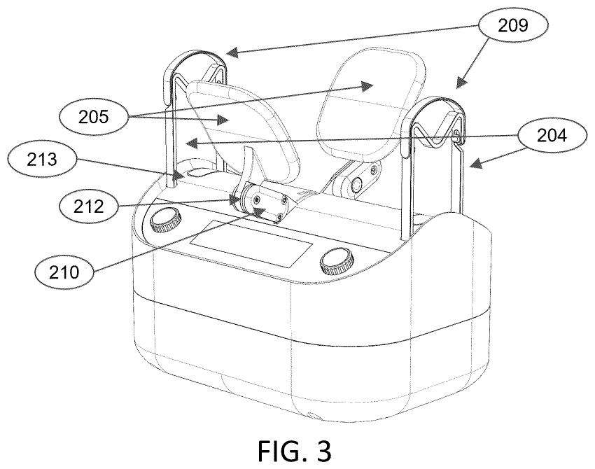

FIG. 3 shows additional aspects of the embodiment depicted in FIG. 2. Folding the paddles 205 about a releasable joint 212 may reduce the system volume during transport by allowing the paddles to lie along the side of the device.

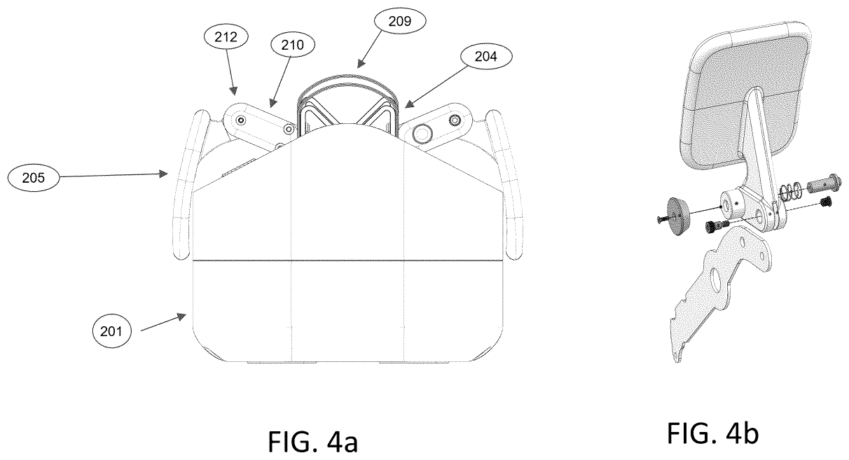

FIG. 4a shows the above-mentioned embodiment with the paddles 205 folded alongside the enclosure 201 by means of folding joint 212. The bag holders 204 and securements 209 have been retracted. FIG. 4b depicts one embodiment of folding joint 212 in which a spring-loaded pull knob may be used to release the arm.

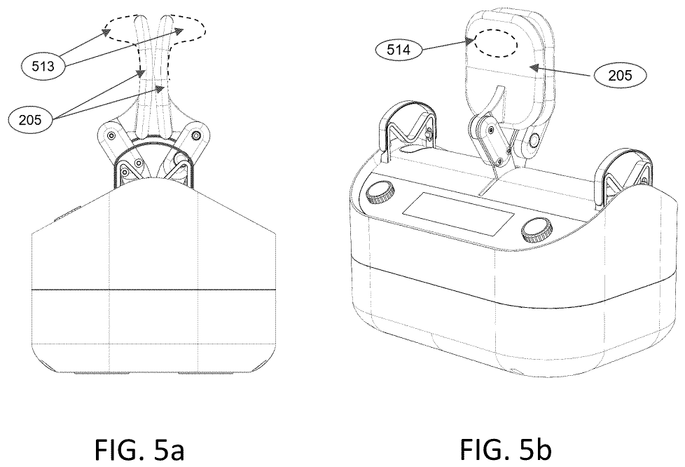

Various possible embodiments would allow the paddles 205 to also serve as handles for carrying the device via a protrusion 513 on the side of the paddle not facing the bag, shown in FIG. 5a or a cutout 514 through the paddles, shown in FIG. 5b. Those skilled in the art will recognize that the inventive carrying configuration of the device may comprise other embodiments which are covered by this disclosure.

### 3. Bag Mounts

Ventilator bags come in a variety of shapes and sizes. In some embodiments the ventilator may allow adjustment of the height and distance of bag mounts to accommodate a range of bag sizes and brands.

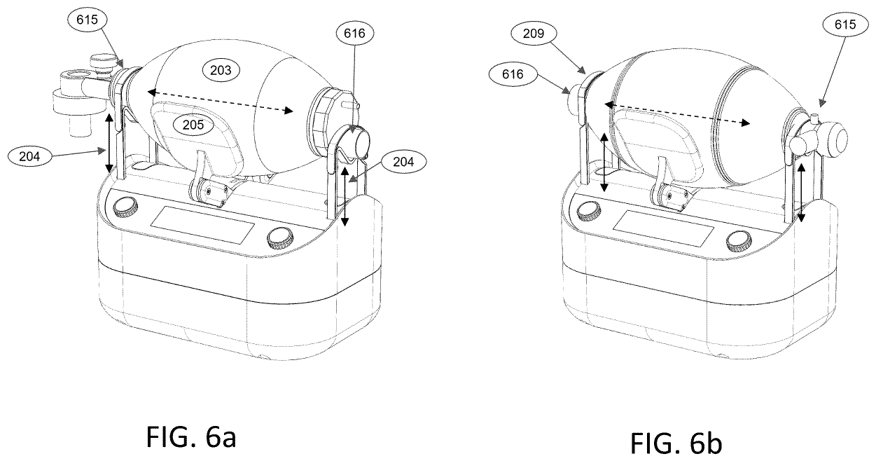

FIGS. 6a and 6b depict exemplary embodiments in which the bag mounts 204 may be adjusted to accommodate bags 203 of different dimensions. Bag mounts raise and lower to accommodate changes in bag dimensions, particularly the anterior 615 and posterior portions 616 so as to maintain the bag's major diameter centered between the paddles 205. Two different bag brands are shown in FIGS. 6a and 6b whereby it is evident that bag's geometries are substantially different. While the shown bag mounts move vertically, bag mounts that are moveable with respect to the long axis of the device and vertically translatable to accommodate a range of bag sizes and brands (e.g., Ambu, Spur). In some embodiments, bag mounts (or bag restraints 209) may move or flex laterally to accommodate the change in bag length as the football-shaped bag is compressed laterally.

### 4. Non-Destructive Actuator

The shape of the paddle may be optimized to minimize or eliminate sliding or rubbing between the resuscitator bag and paddles. The paddles may also be fabricated of or coated with a low-friction material that minimizes or eliminates wear on the bag.

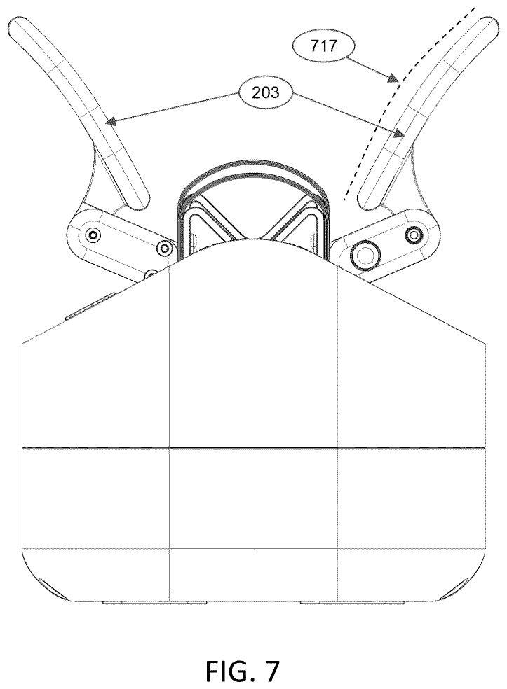

FIG. 7 depicts and embodiment of the devoice in which the shape of the paddle has been optimized to reduce bag wear. The surface 717 of the paddles 203 is shaped so as to reduce wear on the bag by abiding sliding contact. In alternative embodiments, the paddle surface may be shaped to describe a specific breath profile in conjunction with the bag geometry.

### 5. Force-Removable Bag Restraint

In some embodiments, the bag restraints that secure the resuscitator bag in operation will remain secure under normal operating forces, such as during routine activation or transport, but that may release under forces that exceed such normal operating forces. Restraints of this type may also allow the user to readily insert or remove the resuscitator bag, for example to enable a transition between manual and automated bagging.

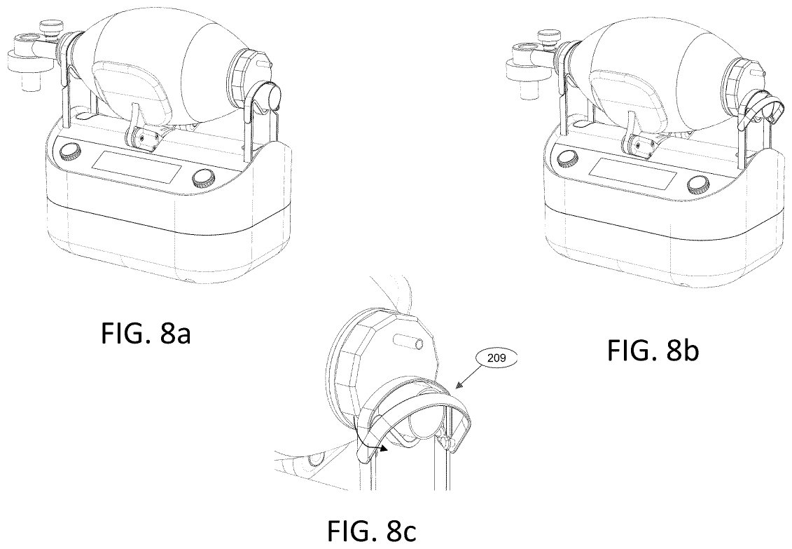

FIG. 8 depicts on such embodiment. In FIG. 8a, the bag is secured by bag restraints 209 as if under normal operation. In FIG. 8b, an excess force has been applied to the bag and the bag restraint has shifted or translated to allow one end of the bag to be removed from the device. FIG. 8c is a closer depiction of the shift or translation of the bag restraint under excess force.

### 6. Control Panel and User Interface

In some embodiments of the device, the enclosure upon which the control panel is located presents a limited amount of space. It may be of increased importance in these embodiments for the operation of the control panel and the user interface to present controls and settings to the user in an efficient manner. The control panel may include a small display and interface that presents critical information to users and allows users to modify certain relevant settings in an effective manner.

Information on the control panel display may be arranged hierarchically, and includes information on presets, adjustments, and alarms. Upon powering up the device, which may be accomplished in some embodiments by pressing the control panel power button, the portable ventilator apparatus and system of the present disclosure may commence artificial respiration immediately, or alternatively with a second button push of the control panel. In such a quick start mode, the second button push may be used to select various default or preset settings for ventilation. For example, the user may select a preset volume, based on estimated weight, or start in pressure support mode (whereby the pressure of oxygen provided builds up to a specified pressure within a specified volume range), or a combination thereof.

In some embodiments, the control panel presents setting information pictographically and numerically such that the interface may be language independent. The portable ventilator apparatus and system of the present disclosure may also include one or more RF transceivers and associated electronics to permit control of the ventilator and data display via wireless communication protocol, such as WiFi, Bluetooth etc., with a smartphone app or other wireless personal electronic device. The device may additionally sound alarms via a connected electronic speaker to guide the user through operation of the apparatus, guide the user to corrective action, or indicate fault conditions.

### 7. Breath Shaping

The portable ventilator apparatus and system of the present disclosure may shape each artificial breath profile by modulating the rate of flow through the gas output port into the patient's lungs. Breath shaping may be accomplished by paddle shape as previously mentioned, but also via control of paddle actuation. Breath-shaping is considered important in normal ventilation conditions, such as in a medical facility, but is considered likely to have benefits in transient situations, especially as the duration of the transient situation increases, such as with longer transport scenarios or when access to a quality medical facility is not readily available. There is currently no way to shape a breath with manual bag actuation.

The shape of each artificial breath is the product of paddle geometry in conjunction with resuscitator bag geometry and the paddle arm's-controlled motion profile. In some embodiments, the ventilator apparatus and system of the present disclosure varies the shape of each artificial breath by varying the speed and acceleration of the paddle arms' motion, thereby achieving breath shaping similar to that of a conventional modern, non-portable ventilator, reducing microstrain on alveoli of the lungs, and minimizing lung injury from mechanical ventilation overpressure.

In order to achieve a specific breath profile and in response to feedback from pressure and flow sensors, some embodiments allow a user to adjust paddle geometry dynamically during operation by manually replacing a paddle of one specific geometry with a different paddle. In some embodiments, the paddle may contain internal mechanisms that adjust its geometry without the need for manual replacement. Paddle geometry and motion profile may also be adjusted to deliver specified breath profiles as a function of the brand and size of the resuscitator bag and different patient profiles. Such patient profiles may include, for example, pre-selected settings for adult, child, and infant patients. Paddle geometry (or actuation) may also be adjusted to permit the portable ventilator apparatus and system's coordination with other healthcare devices as disclosed elsewhere in this disclosure.

### 8. Actuation Mechanism and Drive Train

Various embodiments of the device may use different mechanisms to translate the rotational motion of a motor into the cyclical squeezing of the bag, which could, for example, optimize energy loss or extend lifespan of the motor.

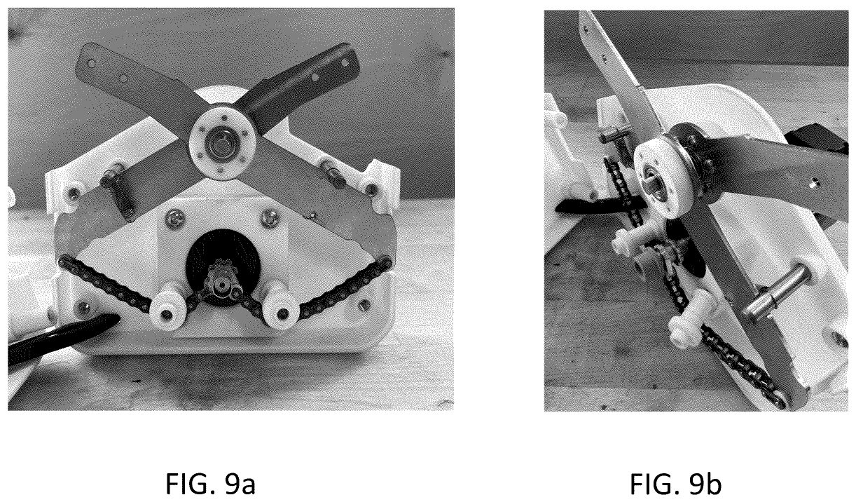

FIG. 9 depicts one such embodiment in which the actuation mechanism consists of a single shaft with two offset sprockets that counter rotates the two paddles together as the shaft rotates. An electric motor is disposed inside the enclosure 201 in a horizontal orientation parallel to the long axis of the enclosure. The motor is attached to a shaft, the other end of which is attached to two chains via offset sprockets. Each chain is attached to the bottom of one of the respective paddle arms 210 which cross diagonally from the bottom through the top of the enclosure. Thus each chain actuates the paddle on the opposite side of the device by means of a scissor-like pivot point. When the motor drives the shaft, the chains engage with the sprockets, pulling the bottom of each paddle arm toward the center of the device and causing the respective arms to move around the pivot point. As the arms move around the pivot point, the paddles move to compress the resuscitator bag. When a desired compression is achieved, the motor reverses direction, allowing the resuscitator bag to elastically return to its undeformed shape and push the paddles into their open positions. A spring connection one or both of the arms to an attachment point within the enclosure may also restore the system to begin another cycle.

### 9. Paddle Force Measurement

In some embodiments, electronics located in the control panel or elsewhere may continuously measure the current draw from the motor and determine or estimate the force exerted by the paddles on the bag and may further derive or infer the pressure within the bag and applied to the subject. In some embodiments this feature may eliminate the need for a mechanical sensor and/or a pressure sensor. In other embodiments deriving pressure from motor current may serve as additional layer of security in the event that other sensors, such as mechanical or pressure sensors, or some other aspect of the system is malfunctioning or otherwise behaving in an unexpected manner. If the pressure sensor fails, or the machine may estimate that there is an “over-pressurization event” occurring because of increased motor current, then it may abort that stroke and alarm to notify the human operator.

In some embodiments, the air pressure of each artificial breath may be determined according to a formula that takes into account the characteristics of the particular brand and size of resuscitator bag being used. A formula relating pressure to bag characteristics may be predetermined, determined during calibration, or determined during operation. In some embodiments, electronics located in the control panel or elsewhere may monitor motor current draw in concert with other sensors to “characterize” the pressures generated when using a given bag.

### 10. Mechanical Fault Detection

In some embodiments, electronics located in the control panel or elsewhere continuously measure the current draw from the motor and monitor for unexpected operation. During normal operation, a motor will draw a predictable amount of current, within some tolerance. When the current actually drawn by a motor falls outside tolerance of the expected amount, a possible fault condition may be inferred. For example, if a motor has encountered a mechanical obstacle which it is trying to overcome, the current draw will increase; conversely, if the mechanical system driven by the motor is providing less resistance (e.g., due to damage or excess compliance of the mechanical system), the current draw will increase.

In some embodiments the device may compare the measured motor current draw against an expected motor current draw, or a range of expected current, for the motor's present state of operation. The device may infer a mechanical abnormality or fault if the current draw exceeds or falls below the expected current draw or range of expected current draw. The device may have a predetermined expected current draw (or range of expected current draw) or may determine such an expected current or range via startup calibration or during operation. The device may further diagnose or classify the probable fault based not only on the fact that current draw is not within expectations, but also based on whether the current is above or below the expected draw and the difference between the observed and expected draw. Inferences about the existence and type of fault may be alerted to the user.

Using current draw to detect possible mechanical faults may eliminate the need for additional mechanical sensors and thereby decrease cost or the complexity of supply chain or maintenance considerations.

### 11. Processing System

The portable, electric, automated mechanical ventilator of the instant disclosure comprises a processing system that may include one or more microprocessors, microcontrollers, field-programmable gate arrays or other programmable logic. The processing system may also include volatile memory, such as RAM, and non-volatile memory, such as Flash or ROM.

The processing system may be operatively connected to the control panel, the actuators for moving the paddles, and current measurement devices. The processing system may be configured to: control the control panel display and receive user inputs from user input controls; shape breaths by controlling the paddle actuator mechanisms; and detect forces applied by the paddles or mechanical faults in the system; in each case as described elsewhere herein. The processing system may also be operatively connected in communication with external sensors and devices, as described below, and configured to automate ventilation, synchronize ventilation with chest compressions, and collaborate with other external devices to deliver CPR. In addition, the processing system may be configured to implement the safety envelopes and features described below.

B. Integrations into CPR Workflow

In another aspect, the ventilation apparatus of this disclosure may be integrated into either a manual CPR workflow to automate the ventilation component of CPR or used in conjunction with other life sustaining equipment such as, for example, a device to automatically deliver chest compressions.

1. Integrate Device into Workflow to Automate Ventilation Component of CPR

The ventilator apparatus of the present disclosure delivers artificial breaths in a controlled manner, at consistent rate and tidal volume, so long as it is supplied with power (either externally or by an internal power source). A first responder manually compressing a resuscitator bag cannot achieve the ventilator apparatus' consistency of breath delivery, nor deliver breaths continuously over long periods without resting or repositioning. Unlike a first responder, the ventilator apparatus of the present disclosure is not susceptible to mental fatigue or the stress inherent in an emergency situation (such as patient cardiac arrest). Human inaccuracy and variability in performing emergency ventilation can result in lung overinflation and barotrauma.

The ventilator apparatus of the present disclosure may be advantageously integrated with chest compressions to improve safety of the CPR process or help supplement delivery of CPR. In an emergency cardiac arrest situation, two trained first responders administer CPR to a patient: one to provide chest compressions, and the other to provide emergency ventilation. The ventilator of the present disclosure may effectively replace one of those two trained first responders in the ventilation component of CPR, and if it is used in concert with an automated chest compression device, a single trained first responder can initiate machine-automated CPR and then be free to conduct other tasks (for example to address other traumatic injury or to help other patients in a mass casualty event).

2. Syncing with Compressions by Activating Based on Direct and/or Indirect Triggers

In a CPR scenario, the ventilator of the present disclosure in certain embodiments may time the delivery of breaths based on chest compressions being delivered to maximize patient air intake and eliminate the need for a pause in chest compressions to accommodate artificial breaths. The instant invention may, for example, initiate the onset of a breath to coincide with the release of a compression or the low point of a patient's inspiration/expiration curve. Currently there is no means to coordinate between chest compressions and breath delivery, other than by manually counting chest compressions and delivering a breath every set number of chest compressions. This requires communication between the responders or manual intervention when an automated chest compression device is used.

In certain embodiments, the ventilator of the present disclosure can detect and count chest compressions by monitoring airway pressure or flow through sensors, which trigger breath delivery every N compressions, where N is a settable or fixed parameter. Alternatively, the ventilator may deliver breaths automatically whenever its sensors detect a pause in chest compressions or, should no chest compressions be detected, default to providing breaths based on a timer in a “sustaining” mode.

The ventilator apparatus of the present disclosure may also, in certain embodiments, coordinate with an unconnected automated external defibrillator (AED), automated compression device or another device through external sensors to detect the compressions or other actions performed upon the patient indirectly, by monitoring airway pressures, chest motions or other parameters, or directly, by measuring the device's action, such as an automated compression device's piston position, motion, or other parameter.

In addition to triggering breaths based on indirect measurements, in some embodiments, the ventilator of the present disclosure may coordinate with a connected AED, automated compression device or another device to initiate breaths based on a digital signal from a connected device. Should no signal be detected, the device may default to a safe, basic ventilation mode or a “sustaining” mode, in which the device provides best efforts to sustain life. Such signals may comprise one or more digital logic-level signals in which each signal may trigger the device on a rising or falling edge; or alternatively, such signals may comprise other communications protocols including, for example, physical or wireless based networking protocols.

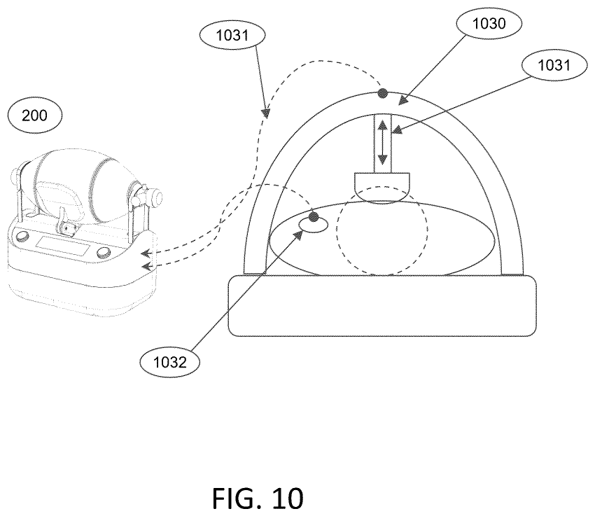

FIG. 10 illustrates certain embodiments wherein the ventilator implements connections with other devices. The device 200 may be connected to different other devices, including, but not limited to an automatic compression device 1030. One or more signals from the automatic compression device may be delivered via a physical connection 1031 or wirelessly. Compressions may be indicated with a digital signal or detected indirectly with a sensor that measures the piston 1032 motion or a sensor 1033 that measures chest motion.

In certain embodiments, the ventilator of the present disclosure may also respond to a direct signal from a user (e.g., the first responder) via a button on the control panel or other input to initiate a breath. Alternatively, the ventilator of the present disclosure may be connected to a data feed or signal from an external chest compression device from which the timing of such chest compressions are directly signaled or may be determined. For example, the ventilator may have a logic level input signaling the beginning, end, or beginning and end of a chest compression via a combination of rising and falling edges. Alternatively, the ventilator may have a wired or wireless communication connection to the chest compression device wherein, for example, the chest compression device sends certain data packets to indicate the timing of its own operation. In any of these scenarios, the ventilator may advantageously be configured to deliver a breath upon detecting a pause in chest compressions.

Coordinate with any other connected device, regarding pausing compressions, checking for rhythm, delivering shocks, resuming CPR, etc.

The processing system of the ventilator may advantageously be configured with a suite of algorithms to automate its methods of interaction with other life support devices or manual CPR.

For example, the processing system may be advantageously configured with an algorithm whereby the ventilator automatically delivers a breath upon detection of a pause in chest compressions, obviating the need for a digital signal from a separate device and, in the case of manual compressions, freeing a first responder from either pausing and restarting the ventilator or manually compressing a resuscitator bag.

As another example, the processing system may be advantageously configured with an algorithm whereby the ventilator, through sensors and external devices previously described, controls external devices through digital signals to initiate AED shocks, automatic chest compressions, and artificial breaths in a coordinated way to maximize patient survivability.

In these embodiments, a user may input information regarding external devices, or alternatively the processing system of the ventilator may be configured to automatically discover and establish communications with connected devices. The user interface may also provide the user options regarding which, if any, of the foregoing algorithms to select and run.

In certain embodiments, the ventilator may adhere to one or more predetermined intersystem communication protocols, which enable the automated resuscitator to supplement and predictably work in concert with other systems (e.g., a system that provides automated chest compressions) in ways other than those envisioned by the preceding sections. In some embodiments, the protocol entails: (1) initiating a breath cycle (for example, in response to a single digital rising or falling edge on a trigger input); (2) pausing or restarting automatically in coordination with another device; and (3) indicating the presence and operating status of one device to another.

In certain embodiments, the ventilator system of the present disclosure may be mechanically connected with one or more life support or medical devices, including, for example, a patient transport device, a patient immobilization apparatus, a chest compression device, etc.

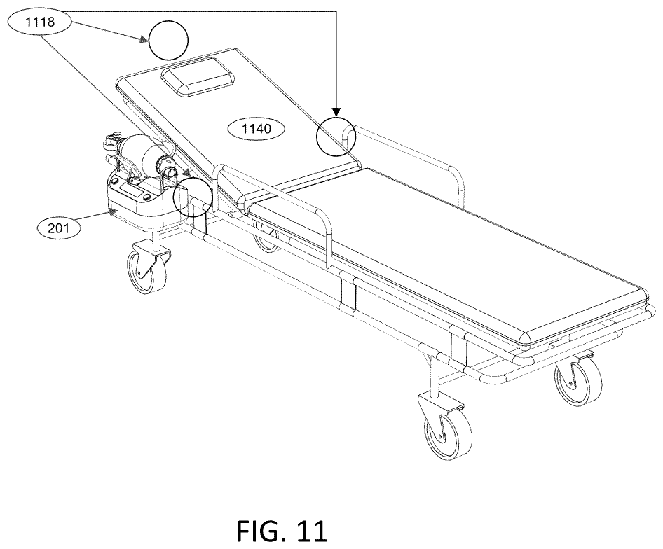

FIG. 11 illustrates one embodiment in which the ventilator may be connected to a patient transport device. In this embodiment, the device is equipped with a clamping or securement mechanism 1118 which will allow the device body 201 to be secured to a wide range of positions on the rails or other convenient points of a stretcher, gurney or similar patient transport device 1140.

A mechanical integration of the ventilator system of the present disclosure with other resuscitation devices such as EleGARD Patient Positioning System and/or LUCAS Device or other patient positioning systems and automated chest compressors advantageously allows operation of multiple devices in a layout that minimizes operational interference across devices.

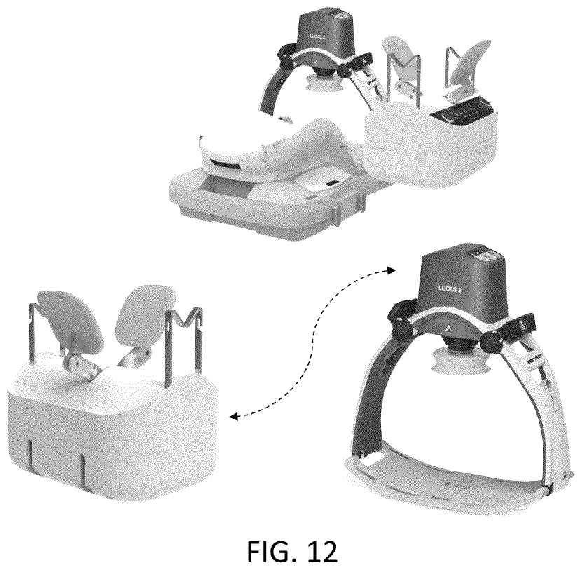

FIG. 12 depicts mechanical interconnections with two exemplary complementary resuscitation and patient support devices. In these embodiments, the ventilator enclosure may feature a snap-on attachment design that allows for simple, fast, and intuitive attachment to minimize training requirements. For certain equipment, including the EleGARD, for example, certain embodiments of the ventilator are configured to facilitate multiple attachment points and positions in the region of a patient's head. In embodiments that include digital integration of the ventilator system with other resuscitation devices, the attachment points may include electrical and data connections. In addition to a mechanical connection, certain embodiments of the ventilator are configured with electrical connectors that are brought into communication with corresponding or mating connectors on the other equipment simultaneously with mechanical attachment. In certain embodiments, the ventilator may share power, control, and user interface systems with the attached equipment. In certain embodiments, the ventilator may rely on such connections, which reduces overall system weight and dimensions.

### C. Define and Enforce Safe Envelopes of Operation to Minimize User Error

In another aspect of the present invention, the ventilator may define and enforce safe envelopes of operation to minimize the chances of a user error occurring or the consequences of such an error. Specifically, in certain embodiments, the instant ventilator may offer conservative preset settings-such as, for example, volume, frequency and pressure safety settings-based on certain parameters-such as, for example, patient weight, age, and condition. Conservative presets may be adjusted if necessary, but may provide an appropriate starting point in a majority of instances. In some embodiments, users may set their own presets based on their patient population, disease profile, or other parameters.

In these embodiments, the control panel may present such conservative preset settings with respect to, among other things, air volume, breath frequency, and pressure safety settings, based on patient weight, age, and other conditions, as input by the user. Users may also use the control pane to set their own presets.

The ventilator apparatus of the present disclosure may additionally measure the pressure and/or volume of delivered air and automatically adjust its operation to maintain pressure and/or volume within a desired envelope of operation. The device may store one or more predefined envelopes comprising, for example, volume of breaths and frequency of breaths, and automatically increase or decrease operational parameters within those predefined envelopes based on or in response to measured patient parameters, such as, for example, end tidal carbon dioxide or airway pressures. Such automatic adjustments may take place within the predefined envelope without intervention from the user. In some embodiments, the feedback loop may be responsive to volume measurement, adjustment and compensation. In addition, pressure safety thresholds and/or volumes may be changed in certain embodiments based on whether compressions are being applied or if the devise is in “sustaining” mode.

The selected operating protocol of the portable ventilator apparatus and system of the present disclosure may be switched manually by the user, or automatically where a change in patient condition or the operation of other resuscitation devices is detected, either through sensors or digital signals from external devices.

The ventilator apparatus of the present disclosure, may in some embodiments, implement modes found only on full-featured fixed ventilators present in medical facilities, thereby extending certain benefits of treatment in a facility into the field or transient ventilation environments.

Traditionally, first responders compress manual resuscitator bags in continuous mandatory ventilation mode (CMV), with minor adjustments made by the first responder based on their direct observation of the patient chest rise and the patient monitor. The ventilator apparatus of the present disclosure, with attached sensors for pressure and flow, enables a much wider range of ventilation modes, increasing the safety and utility of the manual resuscitator and may support longer than normal “manual” ventilation, in the absence of a dedicated transport ventilator

Some examples of modes beyond continuous mandatory ventilation include pressure controlled ventilation (PCV), whereby target ventilation pressure is specified and flow is maintained up to that pressure, and pressure support ventilation (PSV), which allows for patient initiated breaths that the portable ventilator apparatus and system of the present disclosure support by providing additional pressure. The device of the current disclosure may also auto-calibrate respiration, whereby ventilation is started with a low volume and/or a low pressure and the volume and/or pressure is increased up to a preset or stops short based on sensor feedback.

### D. Miscellaneous

The foregoing exemplary descriptions and the illustrative embodiments of the present disclosure have been explained in the drawings and described in detail, with varying modifications and alternative embodiments being taught. While the disclosure has been so shown, described and illustrated, it should be understood by those skilled in the art that equivalent changes in form and detail may be made therein without departing from the true spirit and scope of the disclosure, and that the scope of the present disclosure is to be limited only to the claims except as precluded by the prior art. Moreover, the disclosure as disclosed herein may be suitably practiced in the absence of the specific elements, which are disclosed herein.

1. A portable mechanical ventilator comprising:
a device enclosure housing an electrical subassembly, a mechanical actuation mechanism, a processing system, a control panel, and a power subsystem;
a pair of articulating paddles extending out of the top of the device enclosure, each of which is mechanically connected to the mechanical actuation mechanism via a paddle arm;
a pair of resuscitator bag holders extending out of the top of the device enclosure, each of which comprises an adjustable height riser and a bag securement mechanism; and
a resuscitator bag comprising a gas input port and a gas output port; wherein the resuscitator bag is supported by the pair of articulating paddles, suspended horizontally above the device enclosure, and secured to each of the pair of resuscitator bag holders by means of the bag securement mechanisms; and wherein the mechanical actuation mechanism is configured to actuate each of the articulating paddles simultaneously in a medial direction to compress the resuscitator bag and deliver oxygen to a patient via the gas output port and subsequently in a lateral direction such that the resuscitator bag decompresses and draws in oxygen via the gas input port.

2. The portable mechanical ventilator of claim 1, wherein each of the adjustable height risers is configured to extend the bag securement mechanism away from the device enclosure and retract the bag securement mechanism towards the device enclosure and such extension or retraction is governed by a release button.

3. The portable mechanical ventilator of claim 1, wherein each of the pair of resuscitator bag holders is mechanically connected to the device enclosure with a compliant mechanical connection such that each resuscitator back holder is able to flex laterally to accommodate a change in length of the resuscitator bag as it is compressed.

4. The portable mechanical ventilator of claim 1, wherein each of the bag securement mechanisms is releasable when a force is applied to the resuscitator bag that exceeds a range of force exerted upon the resuscitator bag during a normal operating state of the portable mechanical ventilator.

5. The portable mechanical ventilator of claim 1, wherein each one of the pair of articulating paddles comprises a releasable joint and wherein each one of the pair of articulating paddles is foldable about said releasable joint such that each articulating paddle is disposed along the side of the device enclosure.

6. The portable mechanical ventilator of claim 5, wherein said releasable joint comprises a spring-loaded pull knob that releases the articulating paddle into a foldable state.

7. The portable mechanical ventilator of claim 1, wherein the mechanical actuation mechanism comprises
an electrical motor;
a single drive shaft connected to the electrical motor and comprising two offset sprockets;
two chains, each of which is connected at one end to one of the two offset sprockets and at the other end to a proximal end of one of the pair of paddle arms; and
a spring connection connected to each of the pair of paddle arms;
wherein the paddle arms cross diagonally at a pivot point about which each paddle arm is configured to rotate;
wherein when the motor operates in a first direction to drive the single drive shaft, the chains engage with the sprockets, and pull the proximal end of each paddle arm toward the center of the device and cause each paddle arms to move around the pivot point, which actuates each of the articulating paddles.

8. The portable mechanical ventilator of claim 7, wherein when a desired compression of the resuscitator bag is achieved, the motor operates in a second direction that is the reverse of the first direction, allowing the resuscitator bag to elastically return to its undeformed shape and push the articulating paddles into their lateral positions.

9. The portable mechanical ventilator of claim 1, wherein the processing system
comprises one or more of a microprocessor, a microcontroller, and a field-programmable gate array, one or more volatile memory device, and one or more non-volatile memory device; and
is operatively connected to the control panel, the mechanical actuation mechanism, and a sensor subsystem comprising one or more electrical current measurement devices.

10. The portable mechanical ventilator of claim 9, wherein the processing system is configured to implement a plurality of artificial breath profiles by controlling the mechanical actuation mechanism to modulate a rate of flow through the gas output port.

11. The portable mechanical ventilator of claim 9, wherein the sensor subsystem is configured to monitor an electrical current of the mechanical actuation mechanism.

12. The portable mechanical ventilator of claim 11 wherein the processing system is configured to detect forces applied to the resuscitator bag by the pair of articulating paddles based on said monitored electrical current of the mechanical actuation mechanism.

13. The portable mechanical ventilator of claim 11 wherein the processing system is configured to detect mechanical faults in the system based on said monitored electrical current of the mechanical actuation mechanism.

14. The portable mechanical ventilator of claim 9, wherein the processing system is configured to commence artificial respiration immediately upon entering a powered-on state.

15. The portable mechanical ventilator of claim 14, wherein the processing system is configured to present a plurality of default preset settings via the control panel for said immediate artificial respiration.

16. The portable mechanical ventilator of claim 9, wherein the processing system is configured to
define a safe envelope of operation wherein said safe envelope of operation comprises one or more parameters related to a state of artificial respiration and an acceptable range for each one of said one or more parameters; and
monitor each one of said one or more parameters during artificial respiration.

17. The portable mechanical ventilator of claim 16, wherein said one or more parameters comprise one or more of a volume of gas delivered to the patient, a frequency of actuation, and a pressure of a gas.

18. The portable mechanical ventilator of claim 17, wherein each said acceptable range for each one of said one or more parameters is configurable by a user via the control panel.

19. The portable mechanical ventilator of claim 18, wherein each said acceptable range for each one of said one or more parameters is determined by the processing system based upon data relating to the patient.

20. The portable mechanical ventilator of claim 9, wherein the sensor subsystem is configured to measure a pressure of gas delivered to the patient or a volume of gas delivered to the patient and the processing system is configured to actuate the mechanical actuation mechanism to maintain said pressure or volume within a desired envelope of operation.

21. The portable mechanical ventilator of claim 20, wherein the sensor subsystem is further configured to measure end tidal carbon dioxide or airway pressure and the processing subsystem is configured to automatically adjust actuation of the mechanical actuation mechanism to increase or decrease a volume of gas delivered to the patient or the frequency with which a volume of gas is delivered to the patient.

22. The portable mechanical ventilator of claim 9, further comprising an external signal interface operatively connected to the processing system and configured to receive signals from one or more external devices; wherein the processing system is configured to synchronize operation of the mechanical actuation mechanism with said received signals.

23. The portable mechanical ventilator of claim 22, where said one or more external devices comprises a device configured to automatically deliver chest compressions.

24. The portable mechanical ventilator of claim 23, wherein said received signals indicate a timing of chest compressions and the processing system is configured to time actuation of the mechanical actuation mechanism to deliver gas to the patient at a defined point in a cycle of chest compressions.

25. The portable mechanical ventilator of claim 24, wherein said defined point in a cycle of chest compressions is one of the release of a chest compression or a low point of the patient's expiration.

26. The portable mechanical ventilator of claim 9, wherein the sensor subsystem is configured to monitor airway pressure and the processing system is configured to detect and count chest compressions based on said monitored airway pressure.

27. The portable mechanical ventilator of claim 26, wherein the processing system is configured to detect a state in which chest compressions are paused and automatically actuate the mechanical actuation mechanism to deliver a breath to the patient upon detection of such state.
